(19) United States
(12) Patent Application Publication
(10) Pub. No.: US 2015/0366714 A1
(43) Pub. Date: Dec. 24, 2015

(54) ELECTRONIC EYE MARKING DEVICE

(71) Applicant: Oasis Medical, Inc., Glendora, CA (US)

(72) Inventors: Ganesha Kandavel, Encino, CA (US); James Marlow Christensen, Glendora, CA (US)

(21) Appl. No.: 14/744,841

(22) Filed: Jun. 19, 2015

Related U.S. Application Data
(60) Provisional application No. 62/016,463, filed on Jun. 24, 2014.

(51) Int. Cl. A61F 9/013; A61B 19/00

(52) U.S. Cl. CPC A61F 9/0136; A61B 19/54; A61B 2019/545

(57) ABSTRACT

An electronic eye marking device for marking reference points on a patient's eye in preparation for refractory eye surgery. The device senses its spatial orientation and compares it to the desired target spatial orientation necessary to the patient's eye. The device provides audible and/or visual signals to the user indicating whether the eye marking device is positioned within a predetermined range of the desired target spatial orientation, and it alters the audible and/or visual signals in response to changes in the sensed spatial orientation of the device, thereby providing the user indication of movement closer to, or further from, the desired target spatial orientation.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

The present disclosure relates generally to eye marking devices and methods for use in ophthalmology and optometry procedures. In particular, the present application describes systems, devices, and methods for accurately determining and marking, on a patient's cornea or limbus/conjunctiva, one or more points of a reference meridian in preparation for performing an ophthalmological or optical surgical procedure.

The optical focus of the eye is not always symmetric. The power and proportionate curvature of the cornea, or the total optical system, can be different in different meridians. This introduces the concept of astigmatism, a common type of refractive error wherein the eye does not focus light evenly onto the retina (the light-sensitive tissue at the back of the eye). Refractive surgery is directed at permanently changing the shape of the cornea to restore the eye's focusing power by permitting light rays to focus precisely on the retina. Intraocular cataract surgery lenses that correct astigmatism must be oriented in the correct direction, or axis, in order to correct the astigmatism. It is estimated that each degree of error in the rotational alignment of an implanted toric intraocular lens results in a 3.3 percent loss of the toric correction available to the patient. Accordingly, to avoid residual astigmatism, it is necessary to precisely mark the target axis orientation which serves as a reference meridian during refractive surgical procedures.

Likewise, excimer laser ablation, astigmatic keratotomy, intra-corneal lenses, INTACS™ (inserts including two curved, clear plastic segments that are implanted in the perimeter of the cornea to reduce nearsightedness (myopia) in patients with keratoconus, a visual disorder that occurs when the normally round and dome-shaped cornea progressively thins, causing a cone-like bulge to develop), corneal transplants, spectacle lenses, and any other procedures or therapies that correct astigmatic defocus or wavefront aberrations require the procedure to be performed with the correct orientation on the desired axis. This requires the user or surgeon to have a reference point on the eye with which to align these corrective treatments.

Illustratively, by way of non-limiting example, the surgeon or technician can place markings on the eye (e.g., on the cornea or limbus/conjunctiva) prior to corrective surgery to serve as reference points during the procedure. These marks can be placed at 0 degrees and 180 degrees from the horizontal axis (corresponding to the line of the horizontal axis), 90 degrees and 270 degrees from the horizontal axis (corresponding to the vertical axis), or occasionally on a custom axis, if the marker allows. The patient sits upright during marking, to avoid rotation of the eyes, or cyclotorsion, while recumbent. Commonly a weighted marker or leveled, bubble-dependent, hand-held marking device is used to perform the eye marking. One or more marking tips of the marker are first coated with sterile ink using a surgical marking pen or ink pad. Next the device is leveled using the weight or bubble, and then the patient's eye is marked. The ink is transferred to the cornea or limbus.

Incisional corneal marks have also been used with an orientating mechanism similar to the marker, but rather than marking the tissue with ink, the orienting mechanism micro-perforates the cornea to form a lasting mark. This can also be performed at a slit lamp examination table or in a “free hand” manner by the physician using the marker alone at the bedside. In the same manner, laser vision correction patients or patients receiving INTACS™ or limbal relaxing incisions (manual or with laser cataract surgery) must be oriented for intra-operative accuracy.

Refractive surgical procedures are dependent on accurate preoperative marking. The above-described, previously-available handheld marking devices can be inaccurate and are normally fixed on a specific axis to allow the surgeon to re-mark the eye, intra-operatively, to the desired target axis orientation. Such marking devices are frequently not adjustable or adjustable in a limited manner, and they can be ergonomically challenging. Such devices depend on close observation of visual cues that the marker is on axis, such as a level bubble. Accordingly, the user must simultaneously verify the visual cue and place a mark on the eye, leading to possible movement and inaccuracy during the marking process, as the clinician changes focus between the two.

### SUMMARY

Embodiments of the present disclosure provide an electronic eye marking device for marking reference points on a patient's eye in preparation for refractive eye surgery. The disclosed eye marking device senses its spatial orientation in three dimensions and compares its sensed spatial orientation relative to the desired target spatial orientation for the eye. The disclosed electronic eye marking device provides audible and/or visual signals to the user indicating whether the eye marking device is positioned within a predetermined range of the desired target spatial orientation. The electronic eye marking device alters the audible and/or visual signals in response to changes in the sensed spatial orientation of the device, thereby providing the user indication of movement closer to, or further from, the desired target spatial orientation. The disclosed electronic eye marking device provides the orientation of the marker relative to Earth's gravitational field. It allows the user to hear an audible signal, and based on that signal place a mark on the patient's eye. Advantageously, the disclosed electronic eye marking device frees the user to concentrate on accurate placement of marks on the patient's eye rather than simultaneously visualizing a bubble or weighted handle. Furthermore, the disclosed eye marking device is programmable and adjustable, permitting the marking of any desired target orientation during the initial, pre-operative marking process, and thereby avoiding the need to re-mark the desired angle in a second step, before or during surgery.

Advantages of the disclosed electronic eye marking devices and methods include providing adjustable and customizable eye angle marking with an accuracy to one degree or better; providing audible and/or visual verification signals with respect to the orientation of the marking device, enabling the clinician to concentrate on marking the patient's eye accurately; and providing a means to download to the device individual target spatial data, wavefront aberrometry data, patient health and identity data, and the patient's medical record number, among other information, from another device or system such as, for example, a portable electronic device, an iPad, a personal computer, a hospital monitor, or a central hospital patient data center.

According to one embodiment, the electronic eye marking device emits an audible beeping tone when the device's spatial orientation is different from the target spatial orientation. As the device moves closer to the target spatial orientation, the audible beeping tone is altered, by, for example, increasing the frequency of beeps, or increasing the pitch of the beeping tone, or both. Similarly, when the device moves farther away from the target spatial orientation, the audible beeping sound is altered in a manner indicating that the device is moving farther away from the target spatial orientation, by, for example, decreasing the frequency of beeping, the pitch of the beeping tone, or both. When the electronic eye marking device is positioned within a predetermined range of the desired target spatial orientation, the beeping tone changes, for example, to a continuous tone, thereby indicating to the user that the eye marking device is in the desired spatial orientation angle corresponding to the desired orientation angle on the patient's eye.

In some embodiments, one or more lights (e.g., colored lights) provide visual indications of the relative closeness of the device's spatial orientation to the desired target spatial orientation. The electronic eye marking device emits a flashing red light when the device's spatial orientation is outside of a predetermined range of the target spatial orientation. As the device moves closer to the target spatial orientation, the flashing red light is altered by, for example, increasing the frequency rate by which the light flashes. Similarly, when the device moves farther away from the target spatial orientation, the flashing red light is altered in a manner indicating that the device is moving farther away from the target spatial orientation, by, for example, decreasing the frequency rate by which the light flashes. When the electronic eye marking device is positioned within a predetermined range of the desired target spatial orientation, the light changes color (e.g., changes from red to green) and/or stops flashing, thereby indicating to the user that the eye marking device is in the desired orientation. Advantageously, the one or more lights can be seen by the user's peripheral vision thereby permitting the user to focus visually on the marker relative to the patient's eye.

In an embodiment, the eye marking device includes a handle having a lumen, a distal end, and a proximal end. A marking head is mechanically attached to the distal end of the handle. An electronics assembly is disposed within the lumen of the handle. The electronics assembly includes a processor configured to process sensed information and to control various input and output activity. The electronics assembly also includes a sensor, in communication with the processor and configured to measure a spatial orientation of the handle during use, and to transmit the measured spatial information to the processor. In some embodiments the sensor comprises a three-axis accelerometer. The electronics assembly can further include a speaker and/or one or more lights, both of which are in communication with the processor. The speaker is configured to generate audible sound, and the one or more lights is configured to emit light of at least two different colors.

According to some embodiments, the eye marking device further comprises a shaft, attached to the distal end of the handle and configured to receive the marking head. The shaft may be either permanently fixed to the instrument handle, or it may be rotatable. The Shaft may include a shaft encoder which can serve as a second sensor configured to measure the rotation of the shaft and marking head and to transmit the measured rotation information to the processor. Advantageously, the shaft is configured to receive multiple configurations of the marking head to accommodate the needs of different procedures and the preferences of different clinicians.

The marking head further includes a marker tip which is configured to mark the patient's eye in response to being placed in contact with the eye. In some embodiments, the marker tip communicates with the processor and is further configured to be depressible in response to being placed in contact with the patient's eye. The processor is configured to detect when the marker tip is depressed. In some embodiments, the marker tip triggers an electronic switch when the marker tip is depressed, and the electronic switch transmits a signal to the processor to indicate that that the marker tip has been depressed. In certain embodiments, a pressure sensor is positioned to detect a pressure forced exerted by the marker tip when it is depressed and to transmit a signal to the processor indicating that the marker tip has been depressed. In other embodiments, a force sensor detects the marker tip depression and transmits a signal to the processor. In some embodiments, the processor causes the speaker to emit an audible sound in response to receiving a signal that the marker tip has been depressed, thereby indicating that the marker tip has marked the patient's eye.

In another embodiment, the marking head is removable and interchangeable, permitting various configurations of marking heads to be used with the disclosed eye marking device. In some embodiments, the marking head includes a light positioned centrally to serve as a fixation point for the patient to look at during the marking procedure.

In accordance with an embodiment, the eye marking device includes a display device, positioned on the handle and in communication with the processor. The processor causes the display device to display information during use of the eye marking device. Illustratively, the processor causes the display to present information reflective of the predetermined rotation of the marking head.

In some embodiments, the speaker emits a continuous tone and the light emits a green-colored light when the measured spatial orientation is within a predetermined range of a preselected, desired spatial orientation. The speaker emits a beeping tone and the light emits a red-colored light when the measured spatial orientation of the eye marking device is outside of a predetermined range of the preselected, desired spatial orientation. In some embodiments, the preselected, desired spatial orientation is horizontal relative to Earth's gravity.

In still other embodiments, the electronics assembly further comprises a memory device in communication with the processor and a network interface device also in communication with processor. The network interface device is configured to send and receive data across a network, such as, by way of non-limiting example, a local area network. The eye marking device can receive, from a network by way of the network interface device, patient-related data that may be stored in the memory device. Examples, without limitation, of such patient-related data can include a predetermined spatial orientation, topographic data, wavefront aberrometry data, patient health and identity data, and the patient's medical record number.

According to one embodiment of the present disclosure, a device for marking an eye is disclosed. The device includes a handle having a distal end. The device also includes a marking head, attached to the distal end of the handle and configured to mark the eye in response to being placed in contact with the eye. The device further includes a processor and a sensor in communication with the processor. The sensor is configured to measure a spatial orientation of the handle and to transmit the measured spatial orientation information to the processor. The device for marking an eye further includes a speaker, in communication with the processor and configured to generate audible sounds. The processor, in response to receiving the measured spatial orientation information causes the speaker to emit audible sounds based on the measured spatial orientation information relative to a preselected, desired target spatial orientation.

In an embodiment, the device further includes a light display in communication with the processor and configured to emit light of at least two different colors. The processor, in response to receiving the spatial orientation information causes the light display to emit light of a predetermined color based on the measured spatial orientation information relative to the target spatial orientation.

In accordance with another embodiment of the present disclosure, a method for marking a patient's eye is described. The method comprises inputting into an electronic marking device a target spatial orientation for the patient's eye. The method further includes positioning the electronic marking device near the patient's eye, and sensing a signal from the electronic marking device, wherein the signal changes in response to a sensed spatial orientation of the electronic marking device to provide indication of movement closer to, or further from, the target spatial orientation. The method also includes marking the patient's eye in response to a signal from the electronic marking device indicating that the electronic marking device is positioned within a predetermined range of the target spatial orientation. In some embodiments, the signal emitted from the electronic marking device includes an audible signal. In some embodiments, the signal emitted from the electronic marking device includes a visual signal. The visual signal can comprise colored light, where the colored light is red when the device is not within a predetermined range of the target spatial orientation, and it is green when the device is within a predetermined range of target spatial orientation.

In still another embodiment of the present disclosure, a method of marking a patient's eye is described. The method comprises receiving, by an eye marking device, a target spatial orientation for the patient's eye. The method further includes sensing a spatial orientation of the eye marking device, and emitting a signal, from the eye marking device, responsive to the sensed spatial orientation of the eye marking device. The method also includes changing the emitted signal in response to a change in the sensed spatial orientation of the marking device to provide indication of movement of the eye marking device closer to, or further from, the target spatial orientation. Additionally, the method includes emitting a signal from the eye marking device indicating that the electronic marking device is within a predetermined range of the target spatial orientation, and causing a mark to appear on the patient's eye in response to the eye marking device contacting the patient's eye. In accordance with an embodiment, causing a mark to appear on the patient's eye further includes transferring ink or dye from a marker tip of the eye marking device to the patient's eye. In some embodiments, the emitted signal from the eye marking device comprises an audible signal, a visual signal, or both an audio signal and a visual signal. In some embodiments, the visual signal comprises colored light, wherein the colored light is red when the eye marking device is not within the predetermined range of the target spatial orientation, and the colored light is green when the marking device is within the predetermined range of the target spatial orientation. In still other embodiments, the red light further indicates a direction in which the sensed spatial orientation can be adjusted to approach the target spatial orientation. In still other embodiments, the audible signal further indicates a direction in which the sensed spatial orientation can be adjusted to approach the target spatial orientation by changing frequency. In accordance with certain embodiments of the present disclosure, the emitted signal comprises an audible signal and a visual signal comprising colored light, wherein the colored light is red when the eye marking device is not within the predetermined range of the target spatial orientation, and the colored light is green when the marking device is within the predetermined range of the target spatial orientation. In some embodiments, receiving, by an electronic marking device, a target spatial orientation for the patient's eye further comprises accessing a network and retrieving, over the accessed network, a description of the target spatial orientation for the patient's eye.

For purposes of summarizing the present disclosure, certain aspects, advantages, and novel features of the present disclosure have been described herein. It is to be understood that not necessarily all such aspects, advantages, or novel features can be achieved in accordance with any particular embodiment of the disclosure described herein. Thus the disclosure described herein can be embodied or carried out in a manner that achieves or optimizes one advantage or group of advantages as taught herein without necessarily achieving other advantages as can be taught or suggested herein.

### BRIEF DESCRIPTION OF THE DRAWINGS

Throughout the drawings, reference numbers can be re-used to indicate correspondence between referenced elements. The drawings are provided to illustrate embodiments of the disclosure described herein and not to limit the scope thereof.

FIG. 1 is a schematic perspective view of an electronic eye marking device according to an embodiment of the present disclosure.

FIG. 2A is a schematic top, exploded view of an electronics assembly and a shaft of an electronic eye marking device according to an embodiment of the present disclosure.

FIG. 2B is a schematic side, exploded view of the electronics assembly and shaft of the electronic eye marking device of FIG. 2A.

FIG. 3 is a functional block diagram of an electronics assembly of an electronic eye marking device according to an embodiment of the present disclosure.

FIG. 4 is a flow diagram of a process to mark a patient's eye according to an embodiment of the present disclosure.

### DETAILED DESCRIPTION

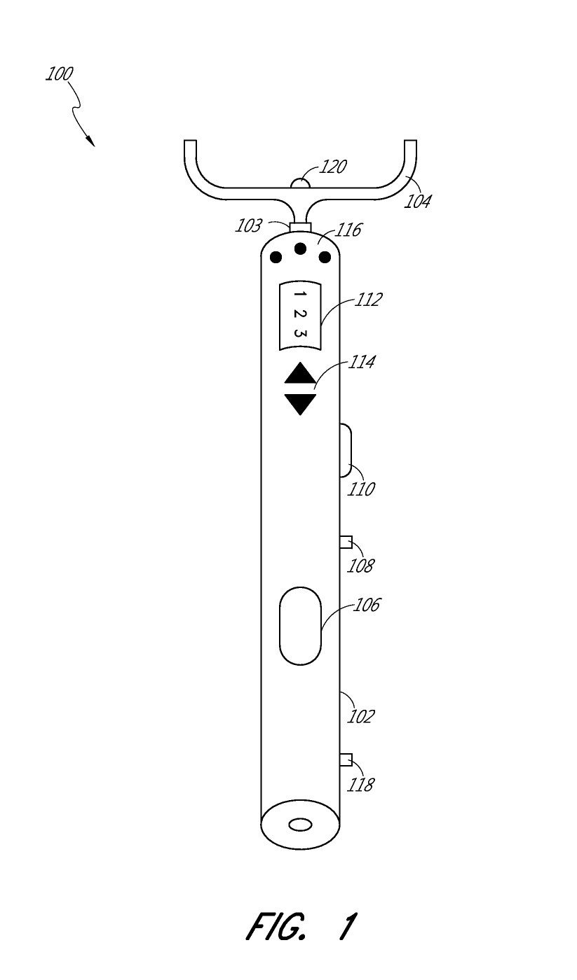

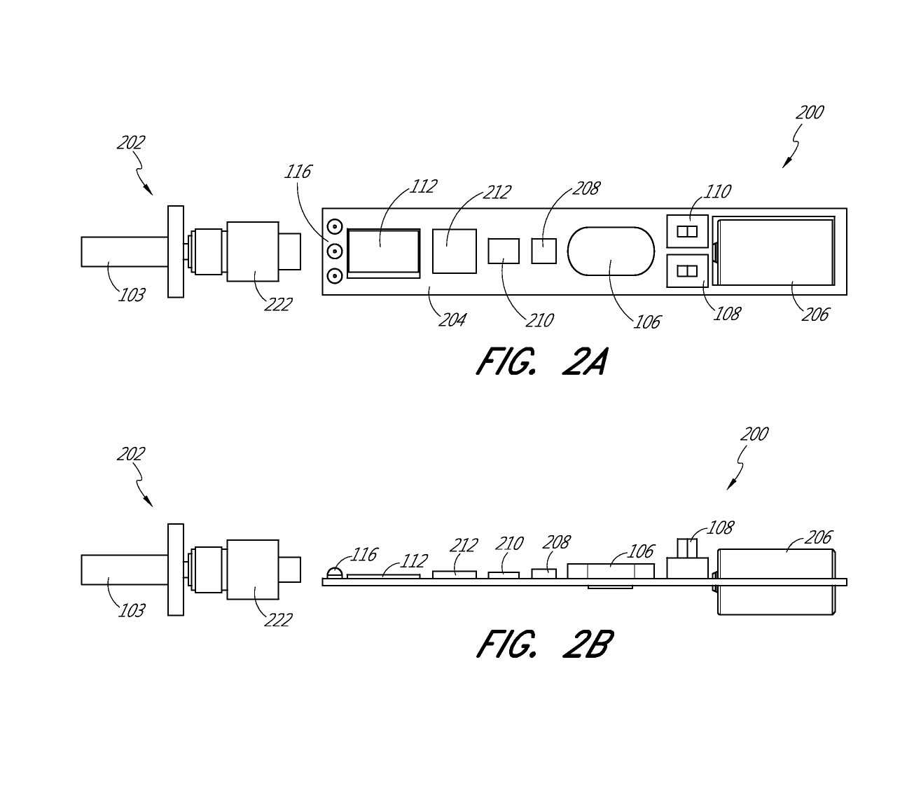

FIG. 1 is a perspective view of an electronic eye marking device 100 according to an embodiment of the present disclosure. The device 100 includes a handle 102, a shaft 103, and a marking head 104. The handle 102 includes a lumen (not shown) in which an electronics assembly (shown in FIGS. 2A-B) is housed. Various controls and displays are connected to the electronics assembly and positioned on the handle 102 including a speaker 106, a power control 108, a speaker volume control 110, a display 112, a marking head rotational control 114, a light display 116, and a system test control 118.

The speaker 106 can provide audible sounds during operation of the electronic eye marking device 100. In accordance with an embodiment, once the desired spatial orientation is selected, and the user is prepared to begin the marking process, a first sound (e.g., an audible beeping) is heard. This audible signal changes (e.g., in rate of beeping and/or in frequency) as the device gets closer to the desired spatial orientation. For example, the beeping continues until the eye marking head 104 is within a predetermined, acceptable range of the desired target orientation at which point, the audible signal changes to a different sound (e.g., a continuous tone). The frequency of the beeps can change to indicate the direction of movement. This allows the user to concentrate on marking the cornea or limbus of the patient's eye while listening to ensure the marks are correctly aligned to the eye. Of course a skilled artisan will understand that there are many different ways to combine audio signals to indicate to the user the spatial orientation of the electronic eye marking device 100 relative to the desired target spatial orientation. In certain embodiments, the electronic eye marking device 100 can communicate with one or more external speakers or cellular devices by wireless modes of communication, such as Wi-Fi (802.11x), Bluetooth, ZigBee, cellular telephony, infrared, RFID, satellite transmission, proprietary protocols, combinations of the same, and the like. In certain embodiments, the electronic eye marking device 100 can communicate with one or more external speakers by way of wired connection, such as by USB or other types of wired interface protocols.

The power control 108 includes a switch that activates and deactivates the device 100. The speaker volume control 110 can include any form of mechanical, electro-mechanical, or electronic device that regulates the volume of sound emitted by the speaker 106.

In an embodiment, the display 112 includes a 0.5 inch, white organic light emitting diode (OLED) display with resolution of 60×32, and displays three digits corresponding to the selected rotational position of the marking head 104, represented in degrees ranging from 0° to 360°. The desired marking head rotational angle can be adjusted to any desired angle. The marking head rotational control 114 (which may be a rotating dial, “up and down arrowed” buttons, or the like) can be used to change the predetermined angle. The desired orientation can be communicated to the user by the display 112, such as an integrated 3-number LED display 112.

In certain embodiments the light display 116 can optionally include a three-LED display in a red, green, red format. Illustratively, by way of non-limiting example, the red lights are illuminated when the device 100 tilt is greater than one degree from horizontal. The green light is illuminated when the device 100 tilt is less than or equal to one degree from horizontal. In some embodiments, only one red light is illuminated indicating the direction in which the orientation needs to be adjusted. Of course, other ranges and/or thresholds can be used to determine whether the device 100 is in the desired position.

In some embodiments, the system test control 118 can perform an automated test to determine whether the electronic eye marking device 100 is functioning properly. Illustratively, by way of non-limiting example, the electronic eye marking device 100 can test the accelerometer 210 and rotational shaft encoder 222 (described below with respect to FIGS. 2A and 2B) to determine whether they are operating within acceptable tolerances for use. Similarly, the functionality of the other electronic and mechanical components of the electronic eye marking device 100 can be tested to provide assurance to the user that the electronic eye marking device 100 is suitable for use with the patient.

The marking head 104 is attached to the handle 102 at the shaft 103 and is used to mark the surface of the eye to allow for orientation reference marks at the desired angle. The marking head 104 can be a fixed head unit, or it can be interchangeable. Interchangeable marking heads 104 allow for the use of many customized/sterilized marking heads 104—having varying prong orientations, angles, widths, and numbers of tips—to be attached (e.g., removably attached) to the handle 102 of the electronic eye marking device 100. The interchangeable marking heads 104 can be removably attached to the shaft 103 using established methods for attaching components to shafts, such as, for example, without limitation, threaded connection, reverse-threaded connection, press-fit connection, pin/slot connection, chuck mechanisms, chain couplings, flex coupling hubs, flex coupling inserts, flexible rubber couplings, jaw coupling hubs, jaw coupling spiders, rigid couplings, a quick connect and disconnect, or the like. Of course, one skilled in the art will appreciate that there are numerous ways to connect the interchangeable marking head 104 to the shaft 103 without departing from the scope of the present disclosure. In certain embodiments, a fixation light 120, positioned in the marking head 104 can be used as a focal point for the patient to look at during the marking procedure.

In some embodiments, the marking head 104 has marker tips (not shown) that include depressible marking surface buttons. In some embodiments, the marking head 104 includes one or more depressible marker tips that communicate with a processor of the device 100. The marker tips can retract in response to being placed in contact with the patient's eye. The processor is able to detect when the marker tip is depressed. In some embodiments, the marker tip triggers an electronic switch when the marker tip is depressed, and the electronic switch transmits a signal to the processor to indicate that that the marker tip has been depressed. In certain embodiments, a pressure sensor is positioned to detect a pressure forced exerted by the marker tip when it is depressed and to transmit a signal to the processor indicating that the marker tip has been depressed. In other embodiments, a force sensor detects the marker tip depression and transmits a signal to the processor. Of course, one skilled in the art will readily appreciate that there are many ways to detect a depression of the marker tip and to transmit a signal to the processor indicating that the marker tip has been depressed without deviating from the scope of the present disclosure. In some embodiments, the processor causes the speaker 106 to emit an audible sound upon such detection, thereby indicating that the marker tip has marked the patient's eye. This would allow the electronic eye marking device 100 to detect and confirm that a mark has been made. The marker tip can be fixed or interchangeable to the handle. It can therefore be customized. The number, design, and angle of the marker tips can be designed as part of a fixed marking head 104 or as part of an interchangeable marking head 104.

FIGS. 2A and 2B illustrate schematic top and side exploded views, respectively, of an electronics assembly 200 and a shaft assembly 202 of an embodiment of the disclosed electronic eye marking device 100. The electronics assembly 200 is sized to fit within a lumen of the handle 102. The electronics assembly 202 includes a circuit board 204, a battery 206, a power control 108, a speaker volume control 110, a speaker 106, a memory device 208, one or more spatial sensors 210 (e.g., an accelerometer), a processor 212, a display 112, and a light display 116. According to an embodiment, the dimensions of the circuit board 204 are optionally 4.3 inches in length and 0.7 inches in width. The battery 206 is optionally a 6-volt lithium manganese cylindrical cell. The battery may be replaceable or rechargeable. Alternatively, the electronic eye marking device 100 may be powered externally by, for example, a USB connection (not shown).

The memory device 208 can store executable instructions (e.g., programs or applications) to execute on the processor 212 as well as data that is collected by the device 100 itself, input by the user, or transferred from another device or system over a network. The memory device 208 can be any type of random-access memory (RAM), including without limitation, dynamic random-access memory (DRAM), static random-access memory (SRAM), and non-volatile random-access memory (NVRAM or Flash memory).

The spatial sensor(s) 210 can determine the three-dimensional spatial orientation of the electronic eye marking device 100 relative to the Earth's gravitational field. It provides the information by which the device 100 determines its spatial position and orientation, for example, relative to horizontal, during the eye marking procedure. According to an embodiment, the spatial sensor 210 is optionally a three-axis accelerometer which provides an accuracy level of 14 bits in the +/−2 g mode, delivering 4069 counts per g (unit of gravity), component identification number MMA8451Q, manufactured by Freescale Semiconductor, Inc., of Austin Tex. In certain embodiments, the spatial sensor(s) 210 for determining the spatial position and orientation of the electronic eye marking device 100 include a capacitive accelerometer (e.g., silicon wafer design) and a three-axis angular rate sensor (gyroscope).

The processor 212 controls the electronic operation of the electronic eye marking device 100. The processor 212 receives input from the sensors and controls and provides outputs to the displays and speaker to drive functional operation of the device. According to an embodiment, the processor 212 is optionally a microcontroller from the MC9S08 series of microprocessors offered by Freescale Semiconductor, Inc., of Austin Tex. Of course, one skilled in the art would understand than many other components can be used to perform the processing and position sensing functions of the device 100 without departing from the scope of the present disclosure.

Advantageously, certain embodiments of the disclosed electronic eye marking device 100 include the ability to sense and detect spatial change in every axis in three-dimensional space including with respect to tilt and rotation. The geometry in modifying the rotation with changing tilt can be calculated in real time while the patient is being marked which increases accuracy. Depending on the distance from the fulcrum of tilt, once an instrument is tilted away from the perfect horizontal orientation there is a corresponding effect on the rotational change at the level of the marking tip for any degree of rotation in the marker shaft. If the marking tip distance from the fulcrum is known and the distance between marking prongs is known, and both are fixed, then the effect of tilt on rotation of the marking head can be accounted for using a geometric calculation or modification. Illustratively, by way of non-limiting example, if the eye marking device is entirely vertical, then any rotation of the device would bring two marking tips closer together and not change the degree of axis. If the marker is entirely horizontal, then any degree of rotation of the device would have a corresponding one-for-one change in the degree of axis. Therefore by having a processor 212 communicating with one or more spatial sensor(s) 210, the disclosed electronic eye marking device 100 can make real-time determinations of the all the degrees of tilt between perfectly horizontal and vertical orientations, which will increase accuracy of the marking.

The shaft assembly 202 is dimensioned to attach to the distal end of the handle 102, and in some embodiments, to connect to the processor 212. The shaft assembly 202 includes a shaft 103 that is able to receive the marking head 104. The shaft assembly 202 is able to mechanically attach to a distal end of the handle 102. The shaft assembly 202 may also include a shaft encoder 222 that measures the rotation of the shaft 103, and therefore the rotation of the marking head 104, and transmits the measured rotation to the processor 212. Alternatively, the shaft 103 may be permanently mounted in the instrument handle 102. In an embodiment, the shaft encoder 222 includes a miniature rotary absolute encoder that reports the shaft 103 position over 360° with no stops or gaps and delivers output in a pulse width modulated digital output format with 12-bit resolution. The shaft encoder 222 component identification number is MA3, marketed by US Digital, of Vancouver, Wash.

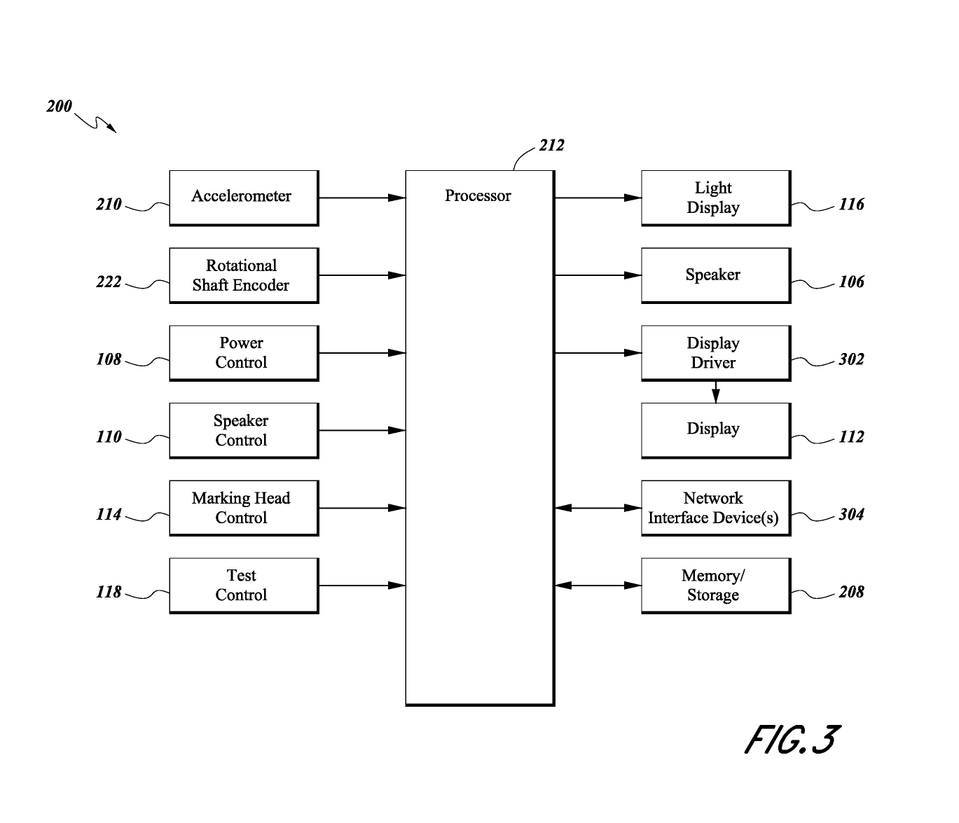

FIG. 3 is a functional block diagram of an electronics assembly 202 of an electronic eye marking device according to an embodiment of the present disclosure. As described above with respect to FIGS. 2A and 2B, the processor 212 controls the electronic functionality of the electronic eye marking device 100. The spatial sensor 210 (depicted in FIG. 3 as an accelerometer), rotational shaft encoder 222 if used, power control 108, speaker control 110, marking head rotational control 114, and test control 118 provide input to the processor 212. The processor 212 executes programs, stored in the memory device 208, and processes the information received from these components to manage and control the functional operation of the device 100. In response to the processed information, the processor causes various actions to be taken by the device 100 as reflected in the outputs the processor 212 transmits to the light display 116, the speaker 106, and the display 112. A display driver 302 receives processor output intended for the display 112 and further process the output to cause the display 112 to present the information to the user.

In an embodiment, the electronics assembly 200 further includes a memory device 208 in communication with the processor 212 and a network interface device 304 also in communication with processor 212. The network interface device 304 is able to send and receive data across a network, such as, by way of non-limiting example, a local area network. The electronic eye marking device 100 can receive, from a network by way of the network interface device 304, patient-related data that may be stored in the memory device 208. Examples, without limitation, of such patient-related data can include a predetermined spatial orientation, topographic data, wavefront aberrometry data, patient health and identity data, and the patient's medical record number.

The network interface device 304 may include any communication device for sending and receiving data across a network, including but not limited to, a network interface card, a modem or another network adapter capable of transmitting and receiving data over a network.

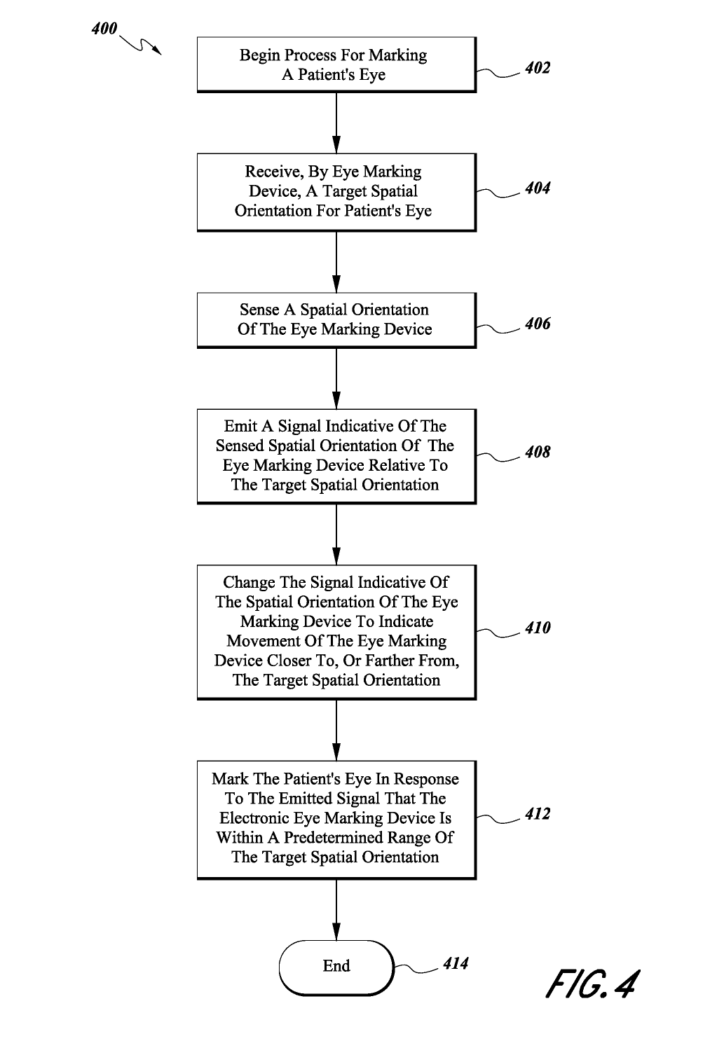

FIG. 4 is a flow diagram of a process 400 to mark a patient's eye according to an embodiment of the present disclosure. The process 400 begins at block 402. At block 404, an eye marking device receives input of a desired target spatial orientation for the patient's eye. In some embodiments, the clinician can enter the target spatial orientation information manually, for example, by way of the marking head rotational control 114. In some embodiments, the target spatial orientation can be input electronically using the network interface device(s) 304. Illustratively, by way of non-limiting example, the eye marking device can retrieve patient-specific information from an electronic medical record system or any other network-based computing or information system. Such patient-specific information can include, among other things, desired target spatial orientation information corresponding to a reference angle to be marked on the patient's eye.

At block 406, the eye marking device begins to sense its actual spatial orientation, as the clinician moves the device toward the patient's eye. At block 408, the eye marking device emits a signal indicating the amount of spatial orientation correction of the device relative to the desired, target spatial orientation. The emitted signal can include an audible signal, a visual signal, and both an audio and visual signal, to name a few. The eye marking device senses its spatial orientation in three-dimensional space and compares its sensed spatial orientation relative to the desired target spatial orientation for the patient's eye. The eye marking device provides audible and/or visual signals to the user indicating whether the eye marking device is positioned in the desired target spatial orientation.

At block 410, the eye marking device alters the audible and/or visual signals in response to changes in the sensed spatial orientation of the device, thereby providing the user indication of movement closer to, or further from, the desired target spatial orientation. The altered signals also indicate the correction needed between the sensed actual spatial orientation of the eye marking device and the target spatial orientation. The eye marking device provides the measured position and orientation of the marker relative to Earth's gravitational field. In some embodiments, the disclosed eye marking device allows the user to hear an audible signal, and based on that audible signal, place a mark on the patient's eye. Advantageously, the eye marking device frees the user to concentrate on accurate placement of marks rather than simultaneously visualizing a bubble or weighted handle. Furthermore, the disclosed eye marking device is programmable and adjustable, permitting the marking of any desired target orientation during the initial, pre-operative marking process, and thereby avoiding the need to re-mark the desired angle in a second step, before or during surgery.

At block 412, the eye marking device emits a signal or signals that indicate that the eye marking device is within a predetermined range of the desired target spatial orientation, thereby indicating that the eye marking device is in position to mark the patient's eye. In some embodiments, the eye marking devices detects when the marker tip(s) are placed in contact with the patient's eye and in response to the detected contact, the eye marking device emits a signal to indicate that the mark has been made. At block 414, the process terminates.

According to another embodiment of the present disclosure, a mobile application (e.g., an iPhone™, Android™, BlackBerry™, or Windows™ mobile application) can be installed on a mobile device, such as a smart cell phone, a tablet, and the like. The user can couple (either wirelessly or through a direct connection) a marking attachment to the mobile device. The marking attachment can connect directly to the mobile device by way of an interface port, such as a USB port, a micro USB port, an Apple™ 30-pin connector port, or the like. The marking attachment can be oriented perpendicular to the display of the mobile device such that the mobile device's display is visible to the user as the marking attachment is directed toward the patient's eye. The mobile application allows the mobile electronic device to use the available hardware, software, interface, and coupled marking attachment, as well as the visual and audio cues available to the mobile device, to perform the functions of the hand-held device 100 for use in the marking operation. Illustratively, in operation, the mobile device can provide visual and/or auditory feedback to the user as the marking attachment is directed to the patient's eye. In some embodiments the marking attachment can rotate to permit the user to select a desired target orientation of the marking attachment. In some embodiments the marking attachment includes a shaft 103 to allow for use of interchangeable marking heads 104 as described above. In some embodiments the shaft 103 includes a shaft encoder 222 that measures the rotation of the shaft 103, and therefore the rotation of the marking head 104, and transmits the measured rotation to the mobile device.

Applications of the presently disclosed electronic eye marking device 100 include a multitude of eye procedures including but not limited to astigmatic keratotomy or limbal relaxing incision, laser and conventional cataract surgery for intraocular lens orientation, laser vision correction, INTACS™, glaucoma surgery, and intra-corneal lenses. Even as newer technologies emerge, such as intra-operative iris registration or aberrometry, surgeons will frequently rely on this type pre-operative marking as a safety measure to ensure accurate intra-operative treatment.

While certain embodiments of the present disclosure have been described, these embodiments have been presented by way of example only, and are not intended to limit the scope of the disclosure. Indeed, the novel methods and systems described herein may be embodied in a variety of other forms. Furthermore, various omissions, substitutions and changes in the form of the electronic eye marking systems and methods of making them described herein may be made without departing from the spirit of the disclosure. The accompanying claims and their equivalents are intended to cover such forms or modifications as would fall within the scope and spirit of the disclosure. Accordingly, the scope of the present disclosure is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. An eye marking device for marking a reference point on a patient's eye, the eye marking device comprising:
a handle having a distal end;
a marking head attached to the distal end of the handle, the marking head configured to mark a patient's eye in response to being placed in contact with the patient's eye;
a processor;
a memory device in communication with the processor;
a sensor, in communication with the processor, the sensor configured to measure a spatial orientation of the handle in three dimensions and to transmit information reflective of the measured spatial orientation of the handle to the processor;
a feedback device, in communication with the processor,
wherein the processor, in response to receiving the information reflective of the measured spatial orientation of the handle, causes the feedback device to provide information indicative of the measured spatial orientation of the handle, and
wherein the information indicative of the measured spatial orientation of the handle is determined by comparing the information reflective of the measured spatial orientation of the handle with a predetermined target spatial orientation.

2. The eye marking device of claim 1, wherein the information indicative of the measured spatial orientation of the handle comprises information indicative of a correction required to the spatial orientation of the handle to approach the target spatial orientation.

3. The eye marking device of claim 1, wherein the feedback device comprises a speaker configured to generate an audible sound wherein the audible sound varies between continuous and pulsed sounds.

4. The eye marking device of claim 1, wherein the feedback device comprises a light configured to emit light.

5. The eye marking device of claim 1, wherein the feedback device comprises a light display configured to emit light of at least two colors.

6. The eye marking device of claim 1, further comprising a network interface device, in communication with the processor, the network interface device configured to send and receive data across a network.

7. The eye marking device of claim 1, further comprising:
a shaft assembly, attached to the distal end of the handle, the shaft assembly comprising:
a shaft configured to receive the marking head; and
a shaft encoder, attached to the shaft and in communication with the processor, the shaft encoder configured to measure a rotational position of the shaft and to transmit measured rotational position information to the processor.

8. The eye marking device of claim 7, further comprising a display device, in communication with the processor, wherein the processor causes the display device to display information reflective of the measured rotational position of the shaft.

9. The eye marking device of claim 7, wherein the marking head further comprises a marker tip configured to mark the patient's eye in response to being placed in contact with the patient's eye,
wherein the marker tip is in communication with the processor, and the marker tip is further configured to be depressible in response to being placed in contact with the patient's eye, and
wherein the processor is configured to detect a depression of the marker tip, and in response to detecting that the marker tip is depressed, the processor is configured to cause the feedback device to provide information indicating that the marker tip has been depressed.

10. The eye marking device of claim 1, wherein the marking head is removably attached to the distal end of the handle.

11. The eye marking device of claim 1, wherein the marking head further comprises a fixation light.

12. The eye marking device of claim 1, wherein the sensor comprises an accelerometer.

13. The eye marking device of claim 3, wherein the audible sound is a continuous tone when the measured spatial orientation of the handle is within a predetermined range of the target spatial orientation, and wherein the audible sound is a beeping tone when the measured spatial orientation of the handle is not within the predetermined range of the target spatial orientation.

14. The eye marking device of claim 4, wherein the emitted light is green when the measured spatial orientation of the handle is within a predetermined range of the target spatial orientation, and wherein the emitted light is red when the measured spatial orientation of the handle is not within the predetermined range of the target spatial orientation.

15. The eye marking device of claim 13, wherein the predetermined range is one degree or less.

16. The eye marking device of claim 14, wherein the predetermined range is one degree or less.

17. The eye marking device of claim 6, wherein the eye marking device receives from the network patient-related information and stores the patient-related information in the memory device.

18. The eye marking device of claim 17, wherein the patient-related information includes at least one of a spatial orientation, topographic data, wavefront aberrometry data, patient identity information, and a patient medical record number.

19. A method of marking a patient's eye, the method comprising:
receiving, by an eye marking device, a target spatial orientation for the patient's eye;
sensing, in three dimensions, a spatial orientation of the eye marking device;
emitting a signal, from the eye marking device, responsive to the sensed spatial orientation of the eye marking device;
changing the emitted signal in response to a change in the sensed spatial orientation of the eye marking device to provide indication of movement of the eye marking device closer to or further from the target spatial orientation;
emitting a signal from the eye marking device indicating that the eye marking device is within a predetermined range of the target spatial orientation; and
causing a mark to appear on the patient's eye in response to the eye marking device contacting the patient's eye.

20. The method of claim 19, wherein the emitted signal from the eye marking device comprises an audible signal.

21. The method of claim 19, wherein the emitted signal from the eye marking device comprises a visual signal.

22. The method of claim 19, wherein causing a mark to appear on the patient's eye further comprises transferring ink from a marker tip to the patient's eye.

23. The method of claim 21, wherein the visual signal comprises colored light, and wherein the colored light is red when the eye marking device is not within the predetermined range of the target spatial orientation, and the colored light is green when the marking device is within the predetermined range of the target spatial orientation.

24. The method of claim 23, wherein the red light further indicates a direction in which the sensed spatial orientation can be adjusted to approach the target spatial orientation.

25. The method of claim 19, wherein the emitted signal comprises an audible signal and a visual signal comprising colored light, wherein the colored light is red when the eye marking device is not within the predetermined range of the target spatial orientation, and the colored light is green when the marking device is within the predetermined range of the target spatial orientation.

26. The method of claim 19, wherein receiving, by an electronic marking device, a target spatial orientation for the patient's eye further comprises:
accessing a network; and
retrieving, over the accessed network, the target spatial orientation for the patient's eye.
